(19) United States
(12) Patent Application Publication
(10) Pub. No.: US 2007/0281352 A1
(43) Pub. Date: Dec. 6, 2007

(54) Peripheral blood mononuclear cells

(76) Inventors: Allan B. Dietz, Chatfield, MN (US); Peggy A. Bulur, Rochester, MN (US); Dennis E. Epps, Jacksonville, FL (US); Gaylord J. Knutson, Rochester, MN (US); Stanimir Vuk-Pavlovic, Rochester, MN (US); Abba C. Zubair, Jacksonville, FL (US)

Correspondence Address: FISH & RICHARDSON P.C., PO BOX 1022, MINNEAPOLIS, MN, 55440-1022, US

(21) Appl. No.: 11/444,137

(22) Filed: May 31, 2006

(51) Int. Cl. C12N 5/08; C12N 5/06

(52) U.S. Cl. 435/325; 435/372

(57) ABSTRACT

This document provides methods and materials relates to peripheral blood mononuclear cells. For example, isolated peripheral blood mononuclear cells as well as methods and materials for obtaining and using peripheral blood mononuclear cells are provided herein.

### BACKGROUND

1. Technical Field

This document relates to peripheral blood mononuclear cells as well as methods and materials for obtaining and using peripheral blood mononuclear cells.

2. Background Information

Peripheral blood mononuclear cells (PBMCs) are routinely used for medical, research, and biomedical purposes. For example, many biological assays such as chemotaxis assays, phenotypic assays, and functional or activation assays involve using isolated human PBMCs. The most common source of human PBMCs for laboratory use has been buffy coats, the cells separated from erythrocytes by centrifugation.

### SUMMARY

This document provides methods and materials relates to peripheral blood mononuclear cells. For example, this document provides isolated peripheral blood mononuclear cells as well as methods and materials for obtaining and using peripheral blood mononuclear cells. As described herein, PBMCs can be obtained from the cells retained in leukocyte reduction system chambers (LRSCs). For example, at least 1×10⁸, 5×10⁸, or 1×10⁹ human PBMCs can be isolated from a LRSC following standard plateletpheresis. PBMCs obtained from the cells retained in a LRSC can produce similar numbers of BFU-E, CFU-GM, and CFU-GEMM colonies as those produced from PBMCs obtained from leukocyte filter eluate (LFE). In addition, the percentages of cells positive for CD3, CD4, CD8, CD14, CD19, and CD56 in the PBMCs isolated from LRSCs and LFEs were indistinguishable. PBMCs isolated from LRSCs can express higher levels of CD69 and CD25 in reaction to staphylococcal enterotoxin B than the cells isolated from LFEs. The source of cells affected neither the yield and purity of immunomagnetically isolated CD3⁺ cells, CD14⁺ cells, and CD56⁺ cells nor the function of T cells, NK cells, and in vitro matured dendritic cells (DCs). PBMCs obtained from LRSCs can have CD14⁺ cells that yield more DCs than those obtained from LFEs. In general, one aspect of this document features a method for obtaining peripheral blood mononuclear cells. The method comprises, or consists essentially of, obtaining a cell population from a leukocyte reduction system chamber and isolating peripheral blood mononuclear cells from the cell population. The peripheral blood mononuclear cells can be human peripheral blood mononuclear cells. The leukocyte reduction system chamber can comprise a post-plateletpheresis leukocyte reduction system chamber. The method can result in obtaining at least 1×10⁸ human peripheral blood mononuclear cells per donor collection or per the leukocyte reduction system chamber. The method can result in obtaining at least 5×10⁸ human peripheral blood mononuclear cells per donor collection or per the leukocyte reduction system chamber. The method can result in obtaining at least 1×10⁹ human peripheral blood mononuclear cells per donor collection or per the leukocyte reduction system chamber. The peripheral blood mononuclear cells, when contacted with staphylococcal enterotoxin B, can express a higher level of CD69 than the level observed in peripheral blood mononuclear cells obtained from leukocyte filter eluate and contacted with staphylococcal enterotoxin B. The peripheral blood mononuclear cells, when contacted with staphylococcal enterotoxin B, can express a higher level of CD25 than the level observed in peripheral blood mononuclear cells obtained from leukocyte filter eluate and contacted with staphylococcal enterotoxin B. The peripheral blood mononuclear cells can comprise CD14⁺ cells that yield more dendritic cells than the number of dendritic cells yielded from CD14⁺ cells of peripheral blood mononuclear cells obtained from leukocyte filter eluate.

In another aspect, this document features isolated peripheral blood mononuclear cells obtained from a cell population retained in a leukocyte reduction system chamber following plateletpheresis. The peripheral blood mononuclear cells can be human peripheral blood mononuclear cells. The peripheral blood mononuclear cells, when contacted with staphylococcal enterotoxin B, can express a higher level of CD69 than the level observed in peripheral blood mononuclear cells obtained from leukocyte filter eluate and contacted with staphylococcal enterotoxin B. The peripheral blood mononuclear cells, when contacted with staphylococcal enterotoxin B, can express a higher level of CD25 than the level observed in peripheral blood mononuclear cells obtained from leukocyte filter eluate and contacted with staphylococcal enterotoxin B. The isolated peripheral blood mononuclear cells can comprise CD14⁺ cells that yield more dendritic cells than the number of dendritic cells yielded from CD14⁺ cells of peripheral blood mononuclear cells obtained from leukocyte filter eluate.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIG. 1A is a photograph of a typical example of an isolated chamber of LRSC. FIG. 1B is a graph plotting the number of PBMCs isolated by density gradient centrifugation from the eluate of the erythrocyte filters (E-Filters), leukocyte filters (L-Filters), or buffy coats from one unit of blood and from the cellular residue in LRSCs following normal plateletpheresis.

FIG. 2 is a graph plotting the percentage of CD4⁺-, CD8⁺-, CD14⁺-, CD19⁺-, and CD56⁺-cells in PBMCs isolated from LRSCs (light columns) and LFEs (dark columns). N=4 for all groups. No difference between analogous cells isolated from LRSCs and LFEs was significant (p>0.05).

FIG. 3 is a graph plotting the number of colonies formed from PBMCs isolated from LRSCs (light columns) and LFEs (dark columns). The numbers of erythroid progenitors (BFU-E), granulocyte/monocyte progenitors (CFU-GM), and granulocyte/erythrocyte/monocyte/megakaryocyte progenitors (CFU-GEMM) were indistinguishable (p>0.05).

FIG. 4A contains two graphs plotting the percentage of either CD25⁺ (top panel) or CD69⁺ (bottom panel) cells isolated from LRSCs (light columns) and LFEs (dark columns) that express CD3⁺-, CD4⁺-, CD8⁺-, CD14⁺-, CD19⁺-, and CD56⁺-cells. Hatching indicates the presence of SEB in the medium. N=4 for all groups. FIG. 4B contains two representative flow cytometric dot plots for PBMCs isolated from LRSCs (upper panels) or LFEs (lower panels). Control cells were incubated without SEB (Control) or with SEB and stained with CD4 and CD25 (left panels) or CD4 and CD69 (right panels). The numbers show the percent of cells in the upper right quadrant, i.e., the cells stained with both respective antibodies.

### DETAILED DESCRIPTION

This document provides methods and materials related to PBMCs. For example, this document provides isolated PBMCs as well as methods and materials for obtaining and using PBMCs. As described herein, PBMCs can be obtained from the cells retained in a LRSC. Examples of LRSCs include, without limitation, those found in Gambro Trima collection devices and Cobe Spectra or other similar devices that use centrifugation to manufacture blood component products. In some cases, PBMCs can be obtained from a LRSC that has been in plateletpheresis. For example, whole blood can be subjected to plateletpheresis using a LRSC. After plateletpheresis, the cells retained in the LRSC can be collected and used as a source to obtain PBMCs. The retained cells can be used directly as a source PBMCs or can be subjected to methods designed to obtain PBMCs. Any method can be used to obtain PBMCs from the cells retained in a LRSC. For example, standard centrifugation techniques such as those described herein can be used to obtain PBMCs. Another example of a method that can be used to obtain PBMCs from the cells retained in a LRSC includes, without limitation, immunomagnetic, antibody-based, isolation of contaminating red blood cells.

Once obtained, the PBMCs can be divided into aliquots of PBMCs. In some cases, the obtained PBMCs can be frozen and stored for future use.

In addition to being used to obtain PBMCs, the methods and materials provided herein can be used to obtain other cell populations such as neutrophils or granulocytes. For example, the cells retained in a LRSC that was used for plateletpheresis can be used as a source of neutrophils or granulocytes.

The invention will be further described in the following examples, which do not limit the scope of the invention described in the claims.

### EXAMPLES

### Example 1

### Cells Retained in Leukocyte Reduction System Chambers Provide an Abundant Source of White Blood Cells

The following was preformed to develop and test the use of cells retained in leukocyte reduction system chambers (LRSCs) after plateletpheresis as an abundant source of white blood cells. On average, four times as many cells were isolated from one LRSC residue than from eluates of leukocyte filters retaining the cells from one unit of blood. The cells isolated from LRSCs were fully viable and functional. Cells from both sources responded to activation with staphylococcal enterotoxin B (SEB), but CD4⁺ T cells isolated from LRSCs expressed higher levels of CD25 and CD69 upon activation. In addition, yields of dendritic cells (DCs) matured from CD14⁺ cells isolated from LRSCs were higher. Thus, the cells retained in LRSCs after plateletpheresis provide an abundant source of viable research-grade leukocytes obtained in compliance with the current blood bank practices.

Blood and platelet donors. Volunteers donated blood at the Division of Transfusion Medicine, Mayo Clinic, Rochester, Minn., in accord with the current regulations by the American Association of Blood Banks and U.S. Food and Drug Administration. Donors were eligible for plateletpheresis if they exhibited at least 150×10⁹ platelets per liter of blood and were free of aspirin for at least 36 hours. Donor's antecubital fossa was cleaned with an iodine tincture, and the vein was accessed with a 16-gauge sterile needle.

Leukocyte collection from whole blood. Whole blood, 500 mL, was collected in less than 15 minutes into a LeukoTrap RCPL triple bag system containing citrate phosphate-2-dextrose (CP2D) anticoagulant (Pall Corp., East Hills, N.J.). During collection, blood was agitated on a CompoGard shaker (Fresenius Hemocare, Redmond, Wash.). The cells were further processed according to the LeukoTrap system manufacturer's guidelines. Briefly, after initial centrifugal separation of erythrocytes and platelet rich plasma, the blood collection set was placed in a plasma extractor. The whole blood bag port was opened to allow platelet rich plasma to flow through the white blood cell filter. Filtration was terminated when erythrocytes contaminated the filter's inlet side. The filter inlet and outlet tubing was sealed, and the filter removed from the set.

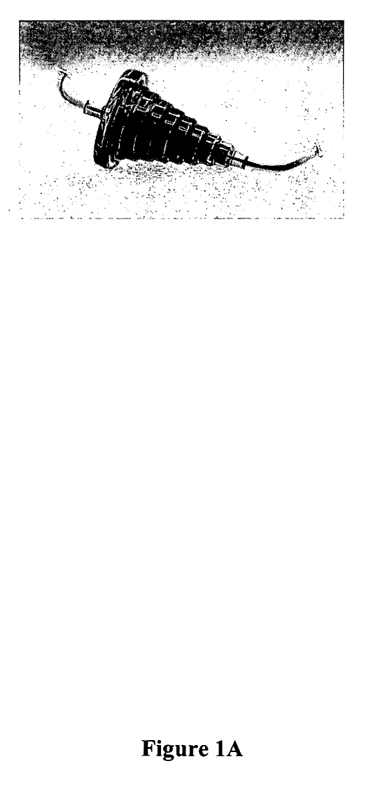

Leukocytes from residue of plateletpheresis. Platelets were collected using a Gambro Trima Accel apheresis apparatus (Gambro BCT, Lakewood, Colo.) controlled by software Version 5.1 with the following settings: anticoagulant management, 4; draw management, 3; return management, 1; maximal draw flow, fast; infusion draw ramp, yes; and anticoagulant ratio, 13:1. Draw rate and return rate were set automatically unless problems in venous access or donor comfort made adjustments necessary. Target yields were 3.0×10¹¹, 3.5×10¹¹, 4.0×10¹¹, 6.2×10¹¹, 6.5×10¹¹, and 6.8×10¹¹ platelets in up to 100 minutes of processing time. Coagulation of the blood and the product was prevented with acid citrate dextrose-A. Once collection had been completed, the platelet collection bag was separated from the disposable set by a heat sealer. The disposable set was removed from the apparatus, and the leads surrounding the LRSC were heat-sealed. The kit was removed and discarded, and the LRSC (FIG. 1A) was stored at room temperature. Within two hours, the tubing was cut at both ends of the LRSC, and the cells were drained into a 50-mL conical tube.

Isolation of peripheral blood mononuclear cells. Leukocyte filters were eluted by gently pushing 50 mL of phosphate-buffered saline (PBS), pH 7.4, in the direction opposite to the one employed at blood filtration. The cells from LRSC were diluted with PBS at the ratio of 1:5. Subsequently, five parts of the undiluted leukocyte filter eluate (LFE) or diluted LRSC cell suspension were layered over two parts of the Lymphoprep solution (ICN Biomedicals, Aurora, Ohio), and the resulting layers were centrifuged at 425×g for 30 minutes at room temperature with no brake applied. The PBMC layer was aspirated and transferred into a 50-mL conical tube, and the cells were collected by centrifugation. The cell pellet was resuspended in PBS and centrifuged at 450×g for 5 minutes followed by a second wash and centrifugation at 300×g for 5 minutes. The cells were resuspended in PBS containing 0.5 percent bovine serum albumin (Sigma-Aldrich, St. Louis, Mo.) and 2.0 mM EDTA (Sigma-Aldrich). A hemocytometer was used to enumerate the cells, and viability was assessed by trypan blue exclusion.

Immunomagnetic isolation of cells. To isolate CD14⁺ cells, 200 μL of CD14-specific immunomagnetic reagent (all immunomagnetic reagents were from Miltenyi Biotec, San Diego, Calif.) were incubated per 4×10⁸ PBMCs. For isolation of T cells and NK cells, the PBMCs were incubated with CD3- or CD56-specific immunomagnetic reagent (at one half of the amount of reagent recommended by the manufacturer). After incubation and washing, the labeled cells were separated on an AutoMACS separator (Miltenyi Biotec) running the POSSEL program. Purity of isolated cells was assessed by flow cytometry using the antibodies listed in Table 1.

TABLE 1
Characteristics of immunoreagents.

| | Fluorescent | |
|---|---|---|
| Antibody specificity | label | Manufacturer |
| CD3 | FITC | Biosource |
| CD3 | PE | Biosource |
| CD3 | APC | eBioscience |
| CD4 | FITC | eBioscience |
| CD8 | FITC | eBioscience |
| CD14 | PE | eBioscience |
| CD16 | PE | Becton Dickinson |
| CD19 | PE | eBioscience |
| CD25 | APC | Pharmingen |
| CD45 | APC | eBioscience |
| CD56 | FITC | Becton Dickinson |
| CD69 | APC | eBioscience |
| CD80 | FITC | Pharmingen |
| CD83 | PE | Immuntech |
| Live/Dead | 7-AAD | Pharmingen |
| Annexin V | PE | Pharmingen |
| IgG | PE | Biosource |

FITC, fluorescein isothiocyanate; PE, phycoerythrin; APC, allophycocyanin; 7-AAD, 7-amino-actinomycin D. All cells were analyzed live except when stained for CD80 and CD83.

Preparation of mature DCs. The cells were matured as described elsewhere (Dietz et al., Cytotherapy, 2004;6(6):563-70; and Dietz et al., J. Hematother. Stem Cell Res., 2000;9(1):95-101). Briefly, in six-well plates, 6.0×10⁶ immunomagnetically purified CD14⁺ cells were seeded in 3.0 mL of X-VIVO 15 medium (Cambrex, East Rutherford, N.J.) containing 1.0 percent pooled human AB serum (HABS; Cambrex), GM-CSF (800 IU/mL; Berlex, Montville, N.J.), IL-4 (1000 IU/mL; R&D Systems, Minneapolis, Minn.), and 1.0 percent penicillin/streptomycin (Gibco, Grand Island, N.Y.). One mL of fresh medium (containing the same components, but with GM-CSF increased to 1600 IU/mL) was added per well on day 3 of incubation. On day 5, the cells were collected by centrifugation and resuspended at 1.0×10⁶ cells/mL in the fresh maturation medium (X-VIVO 15, 1.0 percent HABS, 800 IU/mL GM-CSF, 1000 IU/mL IL-4, 1100 IU/mL TNF-α (R&D Systems), and 1.0 μg/mL prostaglandin E₂ (Sigma-Aldrich)). Non-adherent mature DCs were collected two days later and characterized for viability, yield, and expression of CD80 and CD83.

Cell characterization by flow cytometry. The cells were characterized by flow cytometry with a FACSCalibur flow cytometer (BD Biosciences, San Jose, Calif.) and the fluorophore-conjugated monoclonal antibodies with specificity indicated in Table 1. By multiple immunostaining, CD3⁺CD45⁺ T cells, CD3⁺CD4⁺CD45⁺ T helper cells, CD3⁺CD8⁺CD45⁺ cytotoxic T cells, CD14⁺CD45⁺ monocytes, CD19⁺CD45⁺B cells, and CD56⁺CD45⁺ NK cells were monitored. Cells were incubated with 7-amino-actinomycin D (7-AAD) to exclude dead cells from analysis. Prior to analysis of DCs, the cells were fixed in 1.0 percent paraformaldehyde. For each analysis, one hundred thousand counts were recorded. Data were analyzed with CellQuest software (BD Biosciences). Generally, the PBMC populations were gated on (based on the characteristic patterns of forward and side scatter and the absence of 7-AAD fluorescence) and quantified by binding of specific antibodies.

Activation of lymphocyte subsets. To determine the responsiveness of leukocyte subsets to activation, PBMCs were stimulated with staphylococcal enterotoxin B (SEB), and the effects were measured by the expression of activation markers CD25 and CD69 (McLeod et al., J. Immunol., 1998;160(5):2072-9; and Caruso et al., Cytometry, 1997;27(1):71-6)). The PBMCs were incubated with SEB (1.0 μg/mL in RPMI-1640 medium (Sigma-Aldrich) supplemented with 5.0 percent human AB serum (Sigma-Aldrich) and 1.0 percent penicillin/streptomycin (Gibco)) in a humidified atmosphere of 5 percent carbon dioxide at 37° C. for 18 hours. The cells were collected by centrifugation, stained for CD25 or CD69, stained for antigens characteristic of particular leukocyte subsets, and analyzed by flow cytometry.

In vitro function of T cells, NK cells and dendritic cells. The function of T cells and NK cells purified from the two cell sources were evaluated by measuring the proliferative response to allogeneic mature DCs (MDCs) as model antigen-presenting cells. A mixture of MDCs derived from four donors was plated at 1.0×10⁴ per well in 96-well plates containing X-VIVO 15 medium supplemented with 1.0 percent HABS and 1.0 percent penicillin/streptomycin. One hundred thousand T cells or NK cells were added to wells containing the MDCs in a final volume of 200 μL. The cells were co-incubated for 84 hours. Twelve hours prior to cell collection with a Skatron (Sterling, Va.) semiautomatic cell harvester, [³H]-thymidine (1.0 μCi in 100 μL) was added to each well. Radioactivity incorporated into DNA was measured by a LS 6000SC (Beckman-Coulter, Fullerton, Calif.) scintillation counter. To evaluate the capacity of individual MDC preparations derived from monocytes isolated from the two sources, the same procedure was followed except that CD3⁺ cells were used as responder cells.

Quantifying hematopoietic progenitors. The PBMCs were suspended in MethoCult GF H4434 medium (StemCell Technologies, Vancouver, BC) at final densities of 2×10⁵ per mL. Duplicate 1-mL samples were plated into 35-mm culture dishes and incubated for 14 to 17 days under standard tissue culture conditions. With the aid of an inverted microscope, erythroid colonies (BFU-E; burst forming units-erythrocyte), granulocyte/macrophage colonies (CFU-GM; granulocyte/macrophage), and mixed colonies (CFU-GEMM; granulocyte/erythrocyte/monocyte/macrophage/megakaryocyte) were identified and scored according to StemCell Technologies instructions (Human Colony-Forming Cell Assays Using MethoCult®. Technical Manual. Catalog #28404. Version 3. October 2004. StemCell Technologies).

Statistical analysis. Flow cytometry data represent percentages of live cells labeled by a particular antibody. All data were analyzed by Prism software (GraphPad, San Diego, Calif.), and the significance of differences between and among groups was tested by the two-tailed t-test for unpaired samples or analysis of variance. The probability p<0.05 that the difference was due to chance was taken as significant.

Results

LRS chambers are an abundant source of peripheral blood mononuclear cells. To compare the numbers of PBMCs eluted from filters following filtration of one unit of blood (approximately 450 mL), the erythrocyte and leukocyte filters were cut off from normal donor collections, and 50 mL PBS were passed in the direction opposite to the one used for blood filtering. The numbers of PBMCs obtained from erythrocyte filters, leukocyte filters, and LRSCs were determined (FIG. 1B). The numbers of PBMCs eluted from erythrocyte filters were expectedly negligible, but the numbers eluted from leukocyte filters were high (0.43±0.15×10⁹) and similar to the value reported elsewhere (Meyer et al., J. Immunol. Methods, 2005;307(1-2): 105-66). The slight difference between the two studies may result from the use of larger volumes of sucrose-replete filter-eluting PBS in the Meyer et al. study. The number of PBMCs isolated from LRSCs [(1.88±0.40)×10⁹, n=13] was four times larger than the number of PBMCs isolated from LFEs (0.43±0.15×10⁹, n=8, p<0.0001; FIG. 1B) and twice as large as the number of PBMCs obtained from buffy coats (0.96±0.22×10⁹, n=13, p<0.0001). Although the three methods are not comparable either in the amount of treated blood or in the manner of leukocyte isolation, this result establishes that LRSCs are a useful source of substantial numbers of PBMCs from the hitherto discarded material.

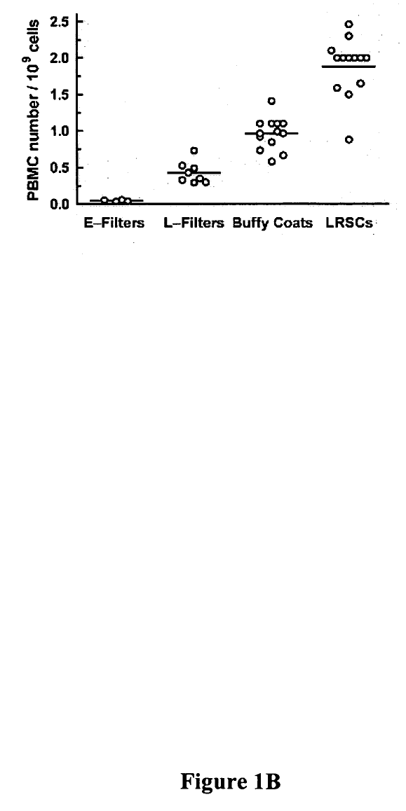

As buffy coats are becoming increasingly unavailable, the PBMCs isolated from LFEs and LRSCs were compared in more detail. Hence, the relative amounts of CD4⁺-, CD8⁺-, CD14⁺-, CD19⁺-, and CD56+-cells were quantified, and no difference between the amounts of analogous cells isolated from the two sources was found (FIG. 2). The results for LFEs are in overall agreement with the results of reported elsewhere (Meyer et al., J. Immunol. Methods, 2005;307(1-2):105-66). The cell composition in LRSC isolates appears fully comparable to the cell composition in LFEs.

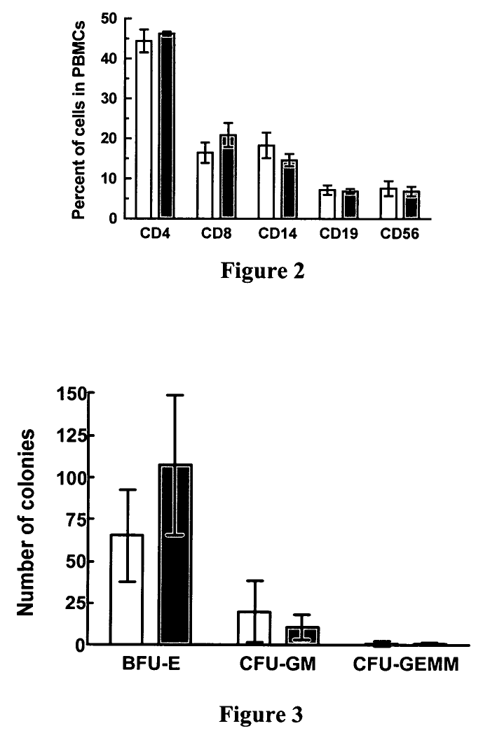

Hematopoietic stem cells and progenitors in PBMCs isolated from LRSCs and LFEs retain similar differentiation potential. The colony formation assay was used to determine the presence of hematopoietic stem cells and early progenitors within the PBMCs. The numbers of BFU-E colonies, CFU-GM colonies, and CFU-GEMM colonies, differentiated from PBMCs prepared from LFEs and LRSCs, were indistinguishable (FIG. 3). In addition, these values were similar to those reported for PBMCs isolated from normal buffy coats employing the same culture conditions (cf. Table 7 in Human Colony-Forming Cell Assays Using MethoCult®. Technical Manual. Catalog #28404. Version 3. October 2004. StemCell Technologies). Thus, LRSCs provide viable hematopoietic stem cells and progenitors in the numbers typically found in PBMCs.

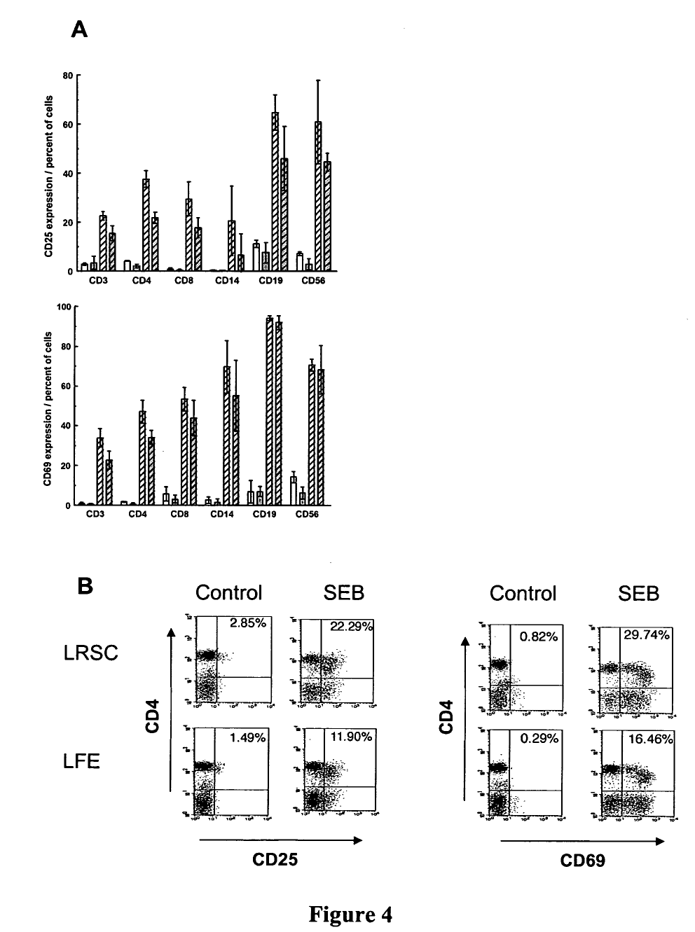

Staphylococcal enterotoxin B activates PBMCs isolated from LRSCs and LFEs. To assess the functional status of major cell populations in the PBMCs isolated from the two white blood cell sources, the PBMCs were incubated with SEB, and the levels of activation markers CD25 and CD69 in the viable CD3⁺-, CD4⁺-, CD8⁺-, CD14⁺-, CD19⁺-, and CD56⁺-cells were measured. SEB strongly affected the levels of CD25 and CD69 in all cells, but the effect was higher in the cells isolated from LRSCs (FIGS. 4A and 4B). The difference in the cell source (LRSCs v. LFEs) accounted for 13 percent of total variance in CD25 (p<0.0001) and 3.7 percent for CD69 (p=0.001). The difference in response among different cell types accounted for the rest. No such differences were observed between control cells from the two sources. Thus, cell subpopulations isolated from LRSCs were fully functional as ascertained by their susceptibility to activation by SEB.

In a more detailed analysis, CD4⁺ T cells isolated from LRSCs were found to respond to SEB by expressing more CD25 (FIG. 4B) and CD69 (FIG. 4D) than the T cells isolated from LFEs (p<0.05; n=4 for all groups). This finding parallels the observation by others that CD4⁺ T cells eluted from filters responded to SEB to a lesser extent than the cells isolated from buffy coats (Meyer et al., J. Immunol. Methods, 2005;307(1-2):105-66). Apparently, filtration and elution affected the potential of CD4+ cells to respond to SEB, while the cells isolated from LRSCs retained their activation potential at levels comparable to the cells from buffy coats. While the isolation method was not found to affect the activation of NK cells, more control (i.e., SEB-free) LRSC-derived NK cells were found to express CD25 and CD69 in comparison to the LFE-borne NK cells (p<0.01; n=4).

PBMCs from LRSCs and LFEs yield highly pure cell subpopulations upon isolation by immunomagnetic adsorption. Immunomagnetic adsorption was used to isolate CD3⁺ cells, CD14⁺ cells, and CD56⁺ cells from PBMCs, and cell yield, purity, and viability were determined. No difference in efficiency of cell isolation from the LFE- and LRSCs-derived PBMCs was observed (Table 2). In addition, the ability of CD3⁺ T cells and CD56⁺ NK cells to synthesize DNA in response to allogeneic MDCs was measured. There was no difference between the cells from the two sources found (Table 3). Thus, all isolated cell populations were highly pure and viable, indicating that the cells isolated from LRSCs and LFEs are similarly amenable to immunomagnetic separation into highly pure and highly viable subpopulations.

TABLE 2
Purity and yield of cells isolated by immunomagnetic selection.

| Specific- | | | | Yield of |
|---|---|---|---|---|
| ity of | | | | isolated cells/ |
| immuno- | Source | Presence in | Purity of | percent of |
| magnetic | of | PBMCs/ | isolated cells/ | respective cells |
| reagent | PBMCs | percent | percent | in PBMCs |
| CD3 | LRSCs | 55.4 ± 1.7 | 99.4 ± 0.2 | 70.2 ± 16.0 |
| | LFEs | 57.7 ± 12.5 | 99.4 ± 0.3 | 65.0 ± 23.7 |
| CD14 | LRSCs | 17.4 ± 5.6 | Not done | 91.0 ± 17.2 |
| | LFEs | 14.3 ± 2.0 | Not done | 98.9 ± 2.4 |
| CD56 | LRSCs | 8.0 ± 4.0 | 90.5 ± 4.9* | 96.0 ± 8.1 |
| | LFEs | 4.9 ± 1.4 | 92.4 ± 3.1 | 89.1 ± 12.7 |

N = 4, except for the group designated by * where n = 3.
LRSCs, leukocyte-reduction system chambers;
LFEs, leukocyte filter eluates.

TABLE 3A
DNA synthesis by CD3⁺T cells and CD56⁺NK cells
isolated from LRSCs and LFEs in response to stimulation
by allogeneic mature dendritic cells.

| | | | [³H]-Thymidine |
|---|---|---|---|
| Stimulator | Responder | Responder cells | incorporated/ |
| cells | cells | isolated from | 1000 × cpm |
| MDC* | CD3⁺T cells | LRSCs | 283.6 ± 83.1 |
| | | LFEs | 286.1 ± 53.2 |
| MDC* | CD56⁺NK | LRSCs | 6.5 ± 3.8 |
| | cells | LFEs | 14.9 ± 15.5 |

*A mixture of equal numbers of mature dendritic cells from eight individuals.

TABLE 3B
Efficiency of mature dendritic cells derived from
CD14⁺cells isolated from LRSCs and LFEs in stimulating
DNA synthesis by allogeneic T cells.

| | | Stimulator cells | |
|---|---|---|---|
| | | matured from | [³H]-Thymidine |
| Stimulator | Responder | CD14⁺cells | incorporated/ |
| cells | cells | isolated from | 1000 × cpm |
| MDC | CD3⁺T cells* | LRSCs | 266.6 ± 100.0 |
| | | LFEs | 238.0 ± 65.5 |

*A mixture of equal numbers of cells from eight individuals.

CD14⁺ cells isolated from LRSCs are a superior source of mature dendritic cells. CD14⁺ cells isolated from LRSCs and LFEs were evaluated for their ability to differentiate into functional MDCs in vitro. The cells were matured, and their yield from CD14⁺ cells and their ability to stimulate the proliferation of allogeneic T cells were measured. After seven days in culture, 29.7±14.6 percent of LRSC-derived CD14⁺ cells matured into DCs (n=7). On the other hand, CD14⁺ cells isolated from LFEs yielded only 10.0±9.1 percent DCs (n=4; p=0.038). This observation is at variance with the data by others who found no difference in DC yields from PBMCs isolated from buffy coats and LFEs (Ebner et al., J. Immunol. Meth., 2001 ;252(1-2):93-104). The reason for the discrepancy may reside in the differences in the composition of the elution buffer, purity of DC precursors, and method of DC culture. Nonetheless, DCs, differentiated from LRSC- and LFE-derived cells, were equipotent in stimulation of allogeneic T cells (Table 3).

In summary, the results presented herein demonstrate that PBMCs isolated from the cellular residue contained in the LRSC following plateletpheresis are a plentiful source of viable and functional leukocytes. This source compares favorably with the cells eluted from the filters introduced recently for leukocyte removal from blood. The advantages of cell isolation from LRSCs are simplicity (as it, unlike isolation from leukocyte filters, requires no elution) and bounty in comparison to the cells isolated from single units of blood.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method for obtaining peripheral blood mononuclear cells, said method comprising obtaining a cell population from a leukocyte reduction system chamber and isolating peripheral blood mononuclear cells from said cell population.

2. The method of claim 1, wherein said peripheral blood mononuclear cells are human peripheral blood mononuclear cells.

3. The method of claim 1, wherein said leukocyte reduction system chamber comprises a post-plateletpheresis leukocyte reduction system chamber.

4. The method of claim 3, wherein said method results in obtaining at least 1×10⁸ human peripheral blood mononuclear cells per donor collection or per said leukocyte reduction system chamber.

5. The method of claim 3, wherein said method results in obtaining at least 5×10⁸ human peripheral blood mononuclear cells per donor collection or per said leukocyte reduction system chamber.

6. The method of claim 3, wherein said method results in obtaining at least 1×10⁹ human peripheral blood mononuclear cells per donor collection or per said leukocyte reduction system chamber.

7. The method of claim 1, wherein said peripheral blood mononuclear cells, when contacted with staphylococcal enterotoxin B, express a higher level of CD69 than the level observed in peripheral blood mononuclear cells obtained from leukocyte filter eluate and contacted with staphylococcal enterotoxin B.

8. The method of claim 1, wherein said peripheral blood mononuclear cells, when contacted with staphylococcal enterotoxin B, express a higher level of CD25 than the level observed in peripheral blood mononuclear cells obtained from leukocyte filter eluate and contacted with staphylococcal enterotoxin B.

9. The method of claim 1, wherein said peripheral blood mononuclear cells comprise CD14⁻ cells that yield more dendritic cells than the number of dendritic cells yielded from CD14⁺ cells of peripheral blood mononuclear cells obtained from leukocyte filter eluate.

10-14. (canceled)
